(19) United States
(12) Patent Application Publication
(10) Pub. No.: US 2008/0183101 A1
(43) Pub. Date: Jul. 31, 2008

(54) Salivary analysis

(76) Inventors: Jonathan Richard Stonehouse, Windlesham (GB); Gordon Robert Davison, Warfield (GB); Donald James White, Fairfield, OH (US); Giuseppe Battaini, Konstanz (DE); Michael James Cannon, Egham (GB)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/891,953

(22) Filed: Aug. 14, 2007

Related U.S. Application Data
(60) Provisional application No. 60/838,221, filed on Aug. 17, 2006.

(51) Int. Cl. A61B 5/00; G06Q 50/00; G06Q 30/00

(52) U.S. Cl. 600/573; 705/2; 705/14

(57) ABSTRACT

The present invention relates to the multivariate analysis of spectra from saliva for estimating the oral health of an individual or group of individuals. The technique enables rapid sampling and evaluation and is particularly useful for facilitating the screening and monitoring of participants in clinical trials, and for evaluating developmental treatment products, as well as providing a straightforward, non-invasive diagnostic method.

### CROSS REFERENCE TO RELATED APPLICATION

This application claims the benefit of U.S. Provisional Application No. 60/838,221, filed Aug. 17, 2006.

### FIELD OF THE INVENTION

The present invention relates to the spectroscopic analysis of saliva, in particular the multivariate analysis of salival spectra. Such analysis is useful for estimating the oral health of an individual or group of individuals or for characterising the effect of treatment products, such as toothpastes or mouth rinses, on the oral environment.

### BACKGROUND OF THE INVENTION

Humans and other animals are susceptible to a range of undesirable oral conditions, such as dental caries, gingivitis and bad breath. Many of these conditions are caused or mediated by bacteria or other micro-organisms within the oral cavity. A wide range of bacteria are normally present in the oral cavity, typically residing as a biofilm on the surfaces of the oral cavity, in particular on the teeth, gums and tongue. Some bacteria or micro-organisms are more harmful than others.

Typically, the undesirable oral conditions start as a low grade, barely detectable disorder which, if left untreated, progresses to a more serious condition. It can be difficult to detect such disorders in their early stages. Whilst doctors and dental professionals are trained in such detection a proper examination is time consuming. Furthermore, even for a trained professional, quantification of the degree of disorder is difficult and an element of subjectivity in the assessment can lead to poor reproducibility. It is particularly a problem for assessing the progression or remission of the disorder within an individual over time. As a consequence, when evaluating products for treating such disorders, reliable clinical trials typically require large base sizes and may need to be run for several months in order to be able to detect differences between products, even though such differences may be clinically important. Other factors affecting such evaluations include a high degree of variability between subjects, relative scarcity of individuals suitable for participating in trials and, whilst the trial is being run, deviation from the desired protocol by individual participants, such as omitting to use, or incorrectly using a treatment product. All of this makes clinical trials very expensive to run which in turn acts as a brake upon the development of improved treatment products.

Much effort has been put into improved methods for assessing oral health. A simple and well know example of assessing the state of the oral cavity is the use of a plaque disclosing table for dyeing, and thereby revealing the extent of, bacterial plaque on the teeth. Whilst the test is simple to perform it does not discriminate well between harmful bacteria and others and is not a reliable indicator of disease state.

It has long been recognised that bacterial metabolites can be implicated in oral diseases. For example, Singer and Bruckner reported, in Infection and Immunity, May 1981, pp. 458-463, the cytotoxic properties of butyrate and propionate, both of which are excreted by dental plaque bacteria. Singer also describes, in U.S. Pat. No. 5,376,532, the spectrophotometric analysis of betaglucoronidase levels in gingival crevicular fluid (GCF) as a means of detecting patients at risk of periodontal disease.

Russian patent no. 2 229 130, published 20 May 2004, uses similar findings as a basis for determining oral-cavity microflora disturbances by quantifying short-chain fatty acids (especially acetic, propionic and butyric) in saliva. The disclosed methods promise a more detailed analysis of the various bacterial species populations.

The use of salivary analysis also has a long history. EP 158 796 (Shah et al.) described the use of a colorimetric test for determining peroxidase in saliva samples as a means of detecting the presence of inflammation due to periodontal disease. More recently, JP 2002/181815 described the use of a strip coated with anti-human hemoglobin monoclonal antibody for detecting occult blood in human saliva as a screening test for periodontal disease. In the method described an individual provides a saliva sample by rinsing with a mouthwash and expectorating. The invention of WO 03/083472 also uses the saliva of a subject to assess the risk of periodontal disease, in this case by examining for the presence/absence of a particular protein by gel electrophoresis, and WO 2005/050204 diagnoses periodontal disease risk, using saliva as a specimen, by detecting lactoferrin polypeptide. Further, Denny et al., in US 2003/0040009, report the use of salivary analysis to predict disease risk, particularly dental caries risk, by quantifying the mucins in saliva.

¹H and ¹³C NMR spectroscopy of human saliva has been reported by Silwood et al. in J. Dent. Res. 81(6):422-427, 2002. The authors report the identification of several biomolecules and a high degree of both inter- and intra-variability between subjects in the pattern of biomolecules. Concluding that ‘NMR spectroscopy serves as a powerful technique for the multicomponent analysis of human saliva’ the authors suggest that the technique may be used for tracking the effects of oral health care products on patients with periodontal diseases.

The foregoing disclosures primarily relate to the analysis of specific chemicals in saliva. A technique using small molecule profiles obtained through a variety of analyses, including spectral and chromatographic analysis, is described as ‘metabolomics’ by the authors of WO 01/78652. Here the emphasis is on use of the whole profile, rather than of individual chemical signals, for diagnosing and predicting disease states, predicting an individual' response to a therapeutic agent and for monitoring the effectiveness of a therapeutic agent in clinical trials.

In the past several years the use of ‘metabonomics’, a technique involving multivariate analysis of spectral data, has also received much attention for assessing disease states, notably from Nicholson and co-workers. For example, WO 02/086478 provides a detailed disclosure of spectral analysis, in particular principal components analysis of ¹H NMR spectra, and its use as a diagnostic technique. The publication discloses a long list of disorders to which the technique might be applied, including dental disorders, such as dental caries, gum disease, and gingivitis. The publication further discloses many fluid sample types to which the technique can be applied, including saliva.

WO 03/107270 builds on the metabonomics approach for the metabolic phenotyping of subjects: This patent application describes the application of metabonomics for, inter alia, predicting responses to dosing, selecting a phenotypically homogeneous set of subjects and for facilitating the identification of biomarkers. WO 2004/038602 further describes generalised techniques for data mining in relation to metabonomics data sets. US 2007/0043518 (Nicholson et al.) expands upon the statistical analyses that can be performed upon metabonomic data sets and their use for identifying components of complex systems, such as identifying biomarkers in biological fluids.

Despite the foregoing there remains the need for further improvement in the management of clinical trials, for the development of improved treatment products, particularly for oral care, and for a more structured approach to characterising the effect of treatment products upon the oral environment.

### SUMMARY OF THE INVENTION

The present invention relates to methods of analysing saliva samples, in particular by using spectroscopic, metabonomic analysis of saliva to get a complete picture of an individual's oral biochemistry. For convenience, the methodology will also be referred to herein as ‘Salivary Metabonomics’. The taking of saliva samples is non-invasive and can be done by an individual at home at a convenient time. The samples are easily stabilised and transported and the spectroscopic technique is capable of producing a large amount of data in a form which is amenable to productive further analysis. Without needing to identify particular compounds the technique is able, for example, to differentiate individuals and to track their responses to treatments. Further, by correlating such analysis to a physician's assessment of the oral health of the same individuals a model can be constructed which can be used to obtain an oral health measure for further individuals. The analyses can be conducted with high throughput and low cost. For example, the analysis enables the management of a clinical trial by screening potential participants and tracking, on a daily basis, actual participants. Used as a screening step to identify potential participants the method enables the selection of a more homogeneous group of relevant participants, or selection of individuals with the most consistent day-to-day saliva composition, thereby improving the power of the trial to detect differences between treatment products. Alternatively or additionally, used as a monitoring step during the trial the method enables a more convenient or more sensitive and objective evaluation of product effects as well as detecting whether trial participants are failing to adhere to the prescribed trial protocol. The ability to provide an oral health measure for a particular individual also makes it possible for the technique to be used as a diagnostic aid. Furthermore, the wealth of data provided can, through multivariate analyses such as principle components analysis, be summarised across individuals to provide a product measure which can provide insight into the mechanism of action of treatment products.

The methods herein can be used e.g.
(i) to determine the kinetics of product action e.g. how many product applications or days of treatment are needed to effect a given change in a subject's saliva composition;
(ii) to measure the efficacy of a product by determining the average change in the concentration of key metabolites after product usage,
(iii) to compare differences in the modes of action between different treatment products by e.g. comparing which particular chemical species change upon product usage.

Details about specific changes in salivary metabolites can be provided e.g. propionic acid, butyric acid, or trimethylamine, which are key metabolites which can be used to compare product efficacies.

The saliva analyses used herein, which can be described as ‘salivary metabonomics’, can also be used to understand consumer perception. For example, some consumers experience “morning mouth”, an unpleasant range of tastes and textures upon wake-up. Metabonomic assessment of these subjects will determine whether their perceptions have a real biochemical basis, or exist simply in their minds. In turn, this learning can be used to develop better products (e.g. utilising actives to target the biochemical basis of the consumer perception, where found).

### BRIEF DESCRIPTION OF THE DRAWINGS

FIG. 1 illustrates the detection of samples containing unusually high levels of ethanol;

FIG. 2 shows the results of a Principle Components Analysis, plotting intervention phase samples on the first two components;

FIG. 3 shows the same samples as in FIG. 2 but after reference phase standardisation;

FIG. 4 is a plot of observed vs. predicted phase identifiers from a model according to the invention;

FIG. 5 shows a ‘Velocity of Action’ plot for the control product of Example 2;

FIG. 6 shows a ‘Velocity of Action’ plot for a test product;

FIG. 7 is a plot of observed overall health scores vs. those predicted by a method according to the invention;

FIG. 8 shows the effect subsequent fitting of components has on the magnitude of eigenvectors from models built by a method according to the invention, for a range of oral care treatment products;

FIG. 9 shows average improvement along a health vector from a model according to the invention for the products shown in FIG. 8;

FIG. 10 is a plot of net changes for individuals following usage of one of several treatment products shown in a space defined by two principle components and related to a health vector.

### DETAILED DESCRIPTION OF THE INVENTION

Unless specified otherwise, all percentages and ratios herein are by weight of the total composition and all measurements are made at 25° C.

As used herein ‘physician’ means any trained professional who is qualified to assess oral health, such as a doctor, a dentist or a dental clinician.

As used herein, oral health measures can be used to estimate diseases or conditions directly affecting the oral cavity such as a plaque, calculus, gingivitis, periodontitis or lingual furring or bad breath or they can be indirect measures of diseases or conditions which primarily affect another part of the body but are nevertheless reflected in some change in oral chemistry, such as a gastric disease or diabetes. In the case of indirect measures the reference model against which the saliva samples are evaluated may be constructed by correlating chemical or biochemical analyses of members of a reference population to reference spectra derived from saliva samples from the reference population members.

In a preferred embodiment herein the invention relates to computing a proxy oral health measure for an individual comprising the steps of:
a) collecting a saliva sample from the individual;
b) obtaining an individual spectrum from the individual's saliva sample;
c) comparing the digitised individual spectrum to a reference model stored in a computer memory to compute the proxy oral health measure, wherein the reference model is derived by correlating, especially through multivariate analysis, one or more direct measures of the oral health of each of a plurality of members of a reference population to reference spectra derived from saliva samples from the reference population members, the reference spectra corresponding in type to the individual spectrum.

By a “direct” oral health measure is meant an observation that is generally accepted as being capable of supporting diagnosis of an underlying oral health condition (such as gingivitis or caries). By a “proxy” oral health measure is meant an observation that is not necessarily diagnostic of the condition but is associated with it and can be used in place of the direct measure, albeit with acceptance of a greater degree of error in a resulting diagnosis. Saliva samples can be easily generated by individuals themselves, in the comfort and privacy of their own homes, thus avoiding the need to visit a clinician. Saliva samples can be frozen for storage and, with suitable stabilisation may be delivered by post or courier to a central facility for analysis. As a result, the proxy measure may be easier or less costly to derive than a direct measure and/or may be more readily repeated over several days to improve confidence in the measure. The methods herein can provide a basis for a personalised health assessment. The direct oral health measures herein are preferably selected from: a physician's quantitative assessment of oral health; gingival images; dental images; and machine readings or expert assessment of breath malodour; in each case for each of the members of the reference population. A preferred method of collecting gingival image data, based upon analysis of the gingival margin is disclosed in U.S. application Ser. No. 11/880,908 (Gerlach et al.) and the equivalent PCT application IB2007/052965. Similar imaging methods can be used for the teeth. US 2007/0092061 discloses an image capture device, system and method for use in capturing digital, dental images and WO 97/06505 discloses a caries detection system based upon digital x-ray images. All of these measures can be reduced to digital form for further analysis on a computer, particularly a multivariate analysis.

In another preferred embodiment the invention relates a method of characterising a treatment product comprising the steps of:
a) collecting at least one starting saliva sample from each of a set of individuals;
b) treating the individuals with the treatment product;
c) collecting at least one end saliva sample from each of the individuals;
d) obtaining and digitising spectra from all of the saliva samples and storing the digitised spectra in a database, each spectrum being associated with an individual identifier and with a sample type identifier;
e) performing a multivariate analysis upon the database of spectra to derive one or more treatment vectors associated with the effect of the treatment product upon the set of individuals.

As used herein, the term “spectrum” refers to a set of linked data obtained by a machine measurement upon a single sample and capable of being captured in digital form as an array of data. The plural “spectra” refers to two or more sets of such data. The terms encompass, in addition to nuclear magnetic resonance, infra-red, ultra-violet and mass (NMR, IR, UV and MS) spectra, chromatograms such as those obtained by liquid or gas chromatography or capillary zone electrophoresis. Preferred are NMR spectra and, in particular, ¹H NMR spectra. The methods herein further include running clinical studies with sets of individuals and determining salivary metabolite levels from samples of the individuals' saliva via spectra obtained from the saliva samples. An advantage of the ‘metabonomics’ methods herein is that, though it is possible to identify and measure particular metabolites, an overall picture of the sample can be obtained by analysing data from the spectra without identifying particular metabolites. Indeed better measures can be obtained by using substantially the whole of, or a large proportion of, the information from the spectra. By correlating the spectral data for the saliva of individuals to a physician's quantitative assessment of the oral health, selected aspects thereof, or other direct oral health measures for the same individuals, reference models can be constructed against which further saliva spectra can be compared to derive proxy oral health measures. The physician's quantitative assessments of the individuals can include one or more indices selected from a plaque index, a calculus index, a gingival index, a periodontal index and a lingual furring index. Even without the correlation to the physician's oral health assessments or other direct oral health measures, analysis of the spectra can reveal important information relating to e.g., the effect of treatment products on the oral environment which is typically replete with a complex variety of bacteria and other organisms and their associated metabolites.

Steps in the taking and analysing of saliva samples and of deriving proxy oral health measures, which can be used for estimating a subject's susceptibility to, or degree of, oral disease typically include the following, though it will be appreciated that many variations are possible.

### Determining Oral Histories

Prior to taking part in a metabonomics study, each potential subject is given an oral soft tissue examination by a registered dentist. A patient medical history is recorded, and the subject is asked to read and sign an Informed Consent form.
If the health and medical history of the subject are deemed suitable for the study, and the appropriate study inclusion and exclusion criteria are met, the subject is enrolled on the study.
Saliva may be collected from healthy individuals, or those with oral diseases (e.g. caries, gingivitis, xerostomia).
In a typical study, subjects are first “washed out” for 3 weeks. That is, they are supplied with a good quality, basic toothpaste capable of providing cleaning but not containing antibacterial actives (e.g. Crest®. Cavity Protection) and a specific toothbrush (e.g. Oral B® Indicator 35). The subjects are asked to brush twice per day, as normal, and to refrain from using all other oral care products. The purpose of this step is to eliminate from the oral cavity any residual antibacterial or other actives, which may have been derived from the subjects' usual oral care products.
Next, “baseline” or “reference phase” data are obtained. The subjects provide sets of saliva samples, over e.g. a 2 week period. These samples provide the reference phase readings for salivary metabolite levels, prior to product intervention.
Finally, the subject is “intervened” with an additional or different oral care product, adding it to, or substituting it for, the existing oral care regimen. Saliva samples are collected from the start of intervention, typically for a period of 3-6 weeks (5 saliva samples per week). These samples enable the impact of the product intervention to be tracked, by monitoring changes in salivary metabolite concentrations through time.

### Saliva Collection & Storage

Study subjects are provided with a set of labelled, screw-cap vials (15 ml, graduated). The vials contain 1.0 ml of deionised water, containing 0.9% w/w of sodium fluoride. The NaF acts to prevent further bacterial action after sample collection. Other saliva stabilisers can also be used.
Subjects are typically asked to provide one sample per day, during Monday to Friday of each study week.
Upon wake up, the subject is requested to refrain from oral hygiene procedures, eating or drinking.
The subject measures 2.0 ml of clean tap water into a disposable Pasteur pipette or vial and uses this to thoroughly rinse the oral cavity, for a timed period of 30 seconds. The entire contents of the mouth are then expectorated into the appropriate supplied vial, and the vial sealed. As an alternative, direct collection of unstimulated or stimulated saliva can be used. Collection of “wake-up” saliva is quite important, as it has been found to be the most metabolite rich, due to the restricted sleeping saliva flow bacterial metabolites are not flushed away.
Optionally, a sugar rinse, or other suitable bacterial food, can be used by the subject at bedtime to amplify the sensitivity of the method. Oral bacteria utilise the sugar overnight and generate raised levels of bacterial metabolites. This is analogous to the cysteine rinses sometimes used to amplify halitosis in halimetry studies.
Each study day, the subject delivers the newly collected saliva vial to a central collection site or puts the vial in a freezer for later delivery, say on a once weekly basis.
The vials are immediately deep frozen, typically at −18° C. The vials remain frozen until preparation for analysis. This saliva sampling and storage protocol has been validated, to confirm that the approach fixes the metabolite concentrations in the samples. An advantage of the method is that it negates the need for a subject to have to visit a dental suite for evaluation of oral health by a dentist or to give a micro-mouth swab or inter-proximal sample. This provides for cheaper sample collection, and due to the convenience of the subject only needing to rinse his or her mouth upon waking, it is more likely that subjects can be recruited and retained on studies and it is more likely that subjects will adhere to the study protocol.

### Saliva Preparation for Analysis

On the day of saliva analysis, samples are withdrawn from the deep freeze, and allowed to defrost for one hour.
The subject identities, sample dates and sample volumes are recorded on a log sheet.
The sealed vials are centrifuged for 30 minutes, at 8000 rpm (=6654 G), with temperature in the centrifuge controlled to 20° C.
Immediately after centrifugation, the supernatant liquid is decanted from the spun-down solids, into appropriately labelled screw-top vials. The solids are disposed of.
80 μL of an NMR reference standard is pipetted into an Eppendorf tube. The reference standard is prepared as follows: 17.24 g of sodium phosphate (dibasic) and 10.84 g of sodium phosphate (monobasic) are dissolved in 1 L of deionised water. The pH is adjusted to 7.0, with either NaOH or orthophosphoric acid. 50 mL of this pH 7 phosphate buffer is rotary evaporated to dryness. The salts are redissolved in 50 ml of D₂O, and the solution again rotary evaporated to dryness. The salts are finally redissolved in 50 mL of D₂O, and 40 μL of pyridazine added.
800 μL of centrifuged saliva is added to the Eppendorf tube.
The entire contents of the Eppendorf tube are transferred, via long glass Pasteur pipette, to a 5 mm diameter NMR tube. The NMR tube is then sealed.
NMR, or other spectral analysis, of the saliva samples is carried out within 48 hours of preparation.
A database of the samples is prepared, to include: unique sample identification code, subject code, sample date, volume of sample, treatment stage, subject gender and age.

### Acquisition of NMR Spectra

Standard proton (¹H) NMR spectra with pre-saturation of the water signal are acquired. Typically, the NOESY presat sequence is used, 128 scans with 10 second relaxation delay and an acquisition time of ˜2 s. The spectra are labelled with a unique sample number from the study.
Following acquisition, NMR spectra are processed (typically 0.5 Hz exponential line broadening), phased, baseline corrected and referenced (usually setting the acetate peak to 1.95 ppm). Alternatively, rather than phasing and baseline correcting, the derivative and absolute value of the spectral data are taken and then referenced as above.

### Analysis of NMR Spectra

The NMR spectra, which are typically 32K complex points, are then “binned” in which the total number of spectral points sum are reduced by dividing the spectrum into a given number of bins and summing up the points within the bins. The analyst can choose the width of the bins, the choice of which typically ranges between 2-10 Hz. During the binning process every spectrum is normalised to the size of the signal from the internal standard such that the total integral of the signal from the internal standard in each of the binned spectra are the same. For ¹H NMR spectra, it can be sufficient to use that part of the spectrum with chemical shifts falling between 0.5 to 3.5 ppm. Preferably at least the portion of the spectrum comprising chemical shifts from 0.5 to 4.5 ppm, more preferably 0.5 to 8.6 ppm, is used. It has also been found to be useful to use at least the portion of each spectrum comprising the peaks for propionic acid, butyrate and trimethylamine. Preferably the portion used further comprises the peaks for formate, N-acetyl sugars, lactate, methylamine, and dimethylamine and more preferably further comprises one or more peaks selected from those for methanol, trimethylamine oxide, phenylalanine, choline, histidine, tyrosine, methylguanidine, sarcosine, β-hydroxybutyrate, succinate, pyruvate, iso-butyrate, n-butyrate, leucine, alanine, n-valerate and ethanol.
The binned spectra are then imported into Microsoft® Excel where additional information is added to each spectrum e.g. subject code, date of sample, stage of the study (e.g. pre-post intervention), gender of the subject, age etc. At this stage, an option that may be taken is that the data can be further manipulated by removing the water and the pyridazine internal standard NMR signals from each spectrum. After removal of the water and pyridazine signals, the entire integral for each of the spectra can then be normalised to the same nominal value. Both data sets are then often used in the subsequent multivariate analysis.
The above spreadsheet can then be loaded into a suitable multivariate package e.g. SIMCA-P+™ from Umetrics Inc.
The subsequent analysis can be broken into a number of discrete steps.
Principal components analysis (PCA) is performed on the binned NMR spectra (X data) in order to identify “outliers”—i.e. those data (spectra) which are anomalous and are very different from the overall data set. PCA is essentially a projection method in which a number of latent variables (principal components—PC) are formed from the original variables (points in the NMR spectrum). The first PC tries to account for the largest variation in the data, the second PC the second largest etc. In this way the complexity of a binned NMR spectrum (˜1000 points) can be represented by much fewer PCs (typically 2-10) allowing visual comparison of hundreds of individual samples. The identification of sample outliers is a combination of using statistical tools (“distance to model”, “Hoteling's T2”) and user judgement in terms of rationalising what signals, and hence what reason exists, for the anomalous behaviour. Any outliers that can justifiably be removed from the dataset are removed and the analysis repeated. There may be several iteration loops here in order to achieve a better dataset.
The “loadings” i.e. the combinations of the original variable (points in the original NMR spectra) making up the various PCs, are analysed from the PCA model to ensure that the model so created is based upon real data rather than NMR spectroscopic artefacts. This involves user judgement. Models built on artefacts must be corrected e.g. the signals in the NMR spectra giving rise to the artefacts can be removed from the data e.g. slight chemical shift differences in signals (especially the acetate signal at ˜1.95 ppm which is generally the largest metabolite signal evident) may result in the model being significantly affected. Often a better model is achieved by omitting the acetate signal from the analysis. Alternatively, differences in chemical shifts of a signal can be corrected by forming a new data bin which covers the spread of the chemical shifts for the signal in question.
The PCA models may be used to identify subjects in the oral care trial who have deviated from the trial protocol e.g. identify mouthwash/dentifrice use or food/drink consumption prior to giving the morning saliva sample. These data and/or subjects may then be removed from the trial resulting in a better quality trial. The PCA models may also be used to pre-screen potential panellists and help select those that would be expected to perform better in the trial e.g. (i) those subjects that have more consistent day-to-day saliva composition (e.g. maybe reflecting lifestyle)—hence more likely to be able to measure a product-induced change in the composition of a person's saliva if their saliva composition is inherently more stable or (ii) select and balance control treatment legs of a study on the basis of the levels of key metabolites in a person's saliva.
Once the PCA model is built and outliers and artefacts have been removed, other multivariate analytical methods are applied as necessary:
PLS Discriminate Analysis (PLS-DA). Here, some prior knowledge of the origin of the saliva samples is used to label the samples e.g. saliva taken “before” and “after” product treatment, or in terms of a particular time period of product treatment use e.g. 0-7 days, 7-14 days etc. A series of “dummy Y” variables is then created for all the NMR spectra from the saliva (X data) in which the “label”—e.g. before/after product treatment is designated by the Y variable taking the value 0 or 1. The subsequent PLS-DA analysis ensures the latent variables making up the principal components are such that the PCs focus on class discrimination (e.g. before/after product treatment). In this way, PLS-DA separates classes of samples on the basis of their X-variables (points in the NMR spectra). In this way a PLS-DA model may be used to determine if a product causes an effect on the saliva composition and if so, how fast a product acts to change the saliva composition. Hence, it can be used to compare the kinetics of action between different products. The model can also be used to identify which chemical species (metabolites from microbes) have changed upon product usage. These species can then be quantified from the NMR spectra (using the pyridazine internal standard) and the degree of change in the amount of particular chemical then used to compare the efficacies between different products. If the PLS-DA model was based upon a particular diagnosed disease state e.g. a healthy and diseased population was selected to form the model, it may then also be used to diagnose disease.
PLS or O-PLS. Here a model is built in which the NMR data from the set of saliva samples is correlated to a second dataset e.g. a set of physician assessed health scores for each subject. In this way ¹H NMR spectra from saliva can be used to predict the physician assessed oral health of further individuals and serve as an objective proxy measure of an individual's oral health. These derived oral health measures are easily obtained and can be used to build up oral histories for individuals by providing an oral health measure for each of a plurality of days for the same individual. When the oral health measures and histories are derived in association with treating the subject with a test substance or composition, they can be used to assess the health benefits, efficacy or mechanism of action of a test substance or composition.
SIMCA: Here, the X-data is assigned membership to a particular class (e.g. before/after product usage, degrees of health state) and a model built which can be subsequently used to predict membership of an unknown sample to the defined classes.
For each of the above multivariate approaches, different scaling of the X-data (centred (Ctr), Univariate (UV), Pareto (Par)) of the variables (the bins from the NMR spectra) is tried. Transforming the X-data e.g. by taking the logarithm or negative logarithm of the binned spectra (to ensure normality of the data) is also evaluated. An “orthogonal signal correction” transformation may also be performed in which X-data not correlated to the Y matrix is first removed prior to building the model. The optimum combination of the above is evaluated in terms of maximising the predictive power of the model.
The models so formed are tested for validity/predictive power e.g. by optimising the “Q2 value” which is calculated by omitting a fraction of the data from the analysis, building a model on the remaining data and then predicting where the omitted data falls. By comparing the prediction vs. the known actual values a measure for the predictive power (Q2) can be formed. Alternatively, a random fraction of the data may also be omitted by the operator and the comparison of the predicted vs. actual values performed. A PLS/PLS-DS model can also be checked against a fortuitous correlation by randomly scrambling the X and Y matrix data and checking that the correlation decreases with the number of random scrambles.
In this way, a measure of the model's predictive ability may be derived and the best model arrived at through several iterations.

### Clinical Study Management

As mentioned above, the oral health measure and histories derived from spectral measures of saliva samples can be use to improve running and management of clinical studies. For example, subjects can be selected for a clinical trial based upon the day-to-day consistency of their saliva composition. By choosing subjects with lower day to day variation in saliva composition, that is, by identifying a subset of the subjects with lower day-to-day variation in saliva composition than the average day-to-day variation in saliva composition taken across the set of subjects as a whole, the power of a clinical trial to differentiate between different product treatments can be increased.

Alternate criteria for selecting subjects from amongst a set of candidate subjects can be:
a) the candidates' oral health measures e.g. selecting a set of subjects with poor oral health,
b) levels of selected metabolites as determined from each candidate's spectrum e.g. selecting subjects with high levels of a particular target metabolite; or
c) a composite measure obtained by integrating data from a plurality of peaks in the individual spectra. This may not be an oral health measure in the sense of having been correlated to a physician's assessment but may nevertheless be a broader indicator of a particular oral chemistry than could be derived from a single metabolite level. Such a measure may be e.g., a proxy measure of a particular oral microflora.

As well as selecting subjects for a clinical trial, the oral health measures or other salival spectra derived measures described above can be useful in trials comprising two or more legs, in that subjects within each leg can be chosen in order to balance the oral health measures or metabolite levels of subjects across each of the legs.

A particular advantage of the methodologies herein is that by examining the oral health histories of subjects on the trial, which can be done on a daily basis, indications of non-compliance with the clinical trial protocol, such as using a non-prescribed treatment product or missing a treatment, can be detected. An objective decision can then be taken as to whether to exclude a subject from the trial for non-compliance, thus helping to produce a more valid or more powerful trial.

A particular advantage of the methods herein is that the saliva samples can be taken by subjects themselves at home and delivered to a central collection point relatively quickly and easily. The subsequent analysis of the saliva samples can be done in a high throughput manner at relatively low cost. One aspect of the invention herein therefore is a method of managing a clinical trial comprising the steps of:
a) recruiting a set of individuals who follow a predetermined protocol including a test or placebo oral treatment over a plurality of days;
b) requesting the individuals to sample their own saliva on one or more of the days and to return the saliva samples to a central collection point;
c) obtaining NMR spectra from the samples after their return to the collection point; and
d) deriving one or more measures from the NMR spectra selected from:
   (i) data on the effectiveness of treatments applied to the individuals over the plurality of days; and
   (ii) data on the day to day responses of individuals in the set.

### Other Uses and Methods

Beyond the uses for improving the management of clinical trials, the methods described herein can be used to improve the management of an individual's health. For example an individual could take a sample of saliva as described herein and have it sent to a laboratory for spectral analysis as herein described to generate an oral health measure or oral health history. The oral health measure or history could then, for example, be provided to the individual's physician as an aid to diagnosis of oral health or other disease state reflected in a change in oral chemistry. The information might for example, be used to assist in the prescription of a treatment product for the individual by examining the individual's oral health measure or history as provided herein. The methodology could also be used in a follow up manner by e.g. treating the individual with a treatment product and assessing the individual's oral health history before and after treatment with the product.

The methods herein are certainly useful for measuring the efficacy or mechanism of action of treatment products and therefore have value in product development. Such measurement can include computing a product efficacy measure for the product from the oral health histories of subjects taking part in a clinical trial, or computing a product efficacy measure from product induced compositional changes in the saliva as determined from the saliva spectra, for a set of subjects taking part in a trial. The measurement may include comparing a test product to a reference product. Product efficacy measures thus obtained could of course be useful for generating advertising indicia for a product by associating the product efficacy measure with the product. Such indicia may include differentiating the mode of action of a product from that of a reference product by showing different product-induced compositional shifts in saliva between the tested product and the reference product.

### EXAMPLE 1

### Mode of Action Investigation

Salivary Metabonomics (SM) employing ¹H NMR was used to investigate the Mode of Action (MoA) of two test toothpastes, A and B, relative to a standard, commercial product, C. Product A included triclosan as an antimicrobial agent and Product B included an antimicrobial system comprising both zinc and stannous salts. Product C did not contain an antimicrobial agent. A group of 30 panellists was selected and instructed to use Product C twice a day for a ‘wash out’ period of four weeks. Over the last two weeks of the wash out period (reference phase) the panellists submitted up to 10 lavage saliva samples each, all taken on wake-up on different days. On each sampling day the panellists used a pipette to pour 2 ml of tap water into their mouth; they rinsed for 30 seconds and then expectorated into a fresh centrifuge tube. The tubes contained 1 ml of 0.9% w/v NaF as a preservative and once filled were stored below 0° C. until submission for analysis.

After the reference phase the group was divided into three legs, individuals being balanced across the legs according to the average % propionic acid found in reference phase saliva (determined from the reference phase NMR spectra). One leg was issued with a new tube of Product C as a placebo, a second leg was issued with Product A and the third received Product B. The panellists used their new products for three weeks (intervention phase) and then for a further two weeks (recovery phase) reverted to the Product C used during the wash-out (baseline) period. During these five weeks the panellists continued to provide up to 5 samples a week. Each leg comprised 8-9 panellists and whilst the link between the panellists and the legs was known throughout, during the data acquisition and processing phase the link between product leg and product was not known.

Submitted saliva samples were logged, labelled with a unique identifier and stored in a freezer. When the samples were prepared for analysis they were taken out of the freezer in approximately the order in which they were submitted (independent of leg) and allowed to thaw for 2 hours. When fully melted, the sample volume was recorded and the samples centrifuged for 10 minutes at 8000 rpm and 20° C. The supernatant was then decanted and stored in a new vial labelled with the same identifier.

The NMR sample was prepared by adding 800 μl of the sample and 80 μl of a buffer solution which contained pyridazine as a reference to a new 18 cm long, 5 mm diameter NMR tube. The sample tube was labelled with the same identifier and submitted for ¹H NMR analysis on a 400 MHz Bruker spectrometer. Samples were racked in a 120 place autosampler, in the order in which they were submitted and were run overnight or over a weekend. Typically 30 would be run per night, with about 40 minutes allowed for each loading, locking, shimming and acquisition cycle. Before running the first sample, the machine was calibrated and a standard shim setting selected. The pyridazine triplet at 9.2 was used to assess the quality of the acquisition and, if necessary, sample acquisitions were repeated at the end of the run and the old spectrum file over-written. The spectra obtained were acquired using water suppression.

NMR pre-processing was carried out using Bruker's XWIN-NMR™ software, all samples in a batch were referenced roughly to the acetate peak at 1.95 ppm. Each spectrum then had the same spectral processing macro applied to it (Scheme 1.1).

Scheme 1.1 Pre-processing macro

lb 1
ef
dt
mc
abs

The macro (the commands of which will be understood by users of the software) performs line broadening and a Fourier transform on the spectrum, takes the magnitude of the first derivative of the spectrum and then performs a spectrum base line correction. It has been found that by taking the derivative of the spectra, overall processing speeds are significantly improved which helps in handling large numbers of samples. The technique reduces the likelihood of finding a statistical break based upon broad signals but gives better resolution for small, sharp peaks. It will be understood that as a result it reduces the validity of comparing one peak with another in a spectrum but it is possible to compare the same peak across several spectra.

The processed spectra were then exported to Bruker's AMIX program where they were referenced more accurately to the acetate peak at 1.95 ppm and then binned using the parameters listed in Scheme 1.2.

Scheme 1.2 AMIX binning parameters

BUCKETS
number of buckets = 900
left = 9.400000
right = 0.400000
width = 0.010000
END
SCALE
scale mode = 1
mulitplier = 1.000000
END
NORMALIZATION
left = 9.300000
right = 9.150000
END
INTEGRATION
mode = 0
END
FILE
format = 3
delimiter =
table = 1
access = 1
END

The bin file was then exported and the bin lists were linked to the data recorded about the particular sample and the person who submitted it. All the samples from the entire trial were binned in the same operation.

Data analysis started by normalising the area under the curve between 3.1 and 0.7 ppm to 100 and the bins from 1.995 to 1.905 (attributed to the CH₂ protons in acetate) were then removed to prevent any variations in acetate levels dominating the model. Bins within some ranges were combined to prevent peak shift reducing the power of the models formed. The particular regions are listed in Scheme 1.3. All samples from the same product leg were given an integer identifier in the sample information. All samples were given a second identifier (a phase identifier) which for reference phase samples was equal to the first integer less 0.1, for intervention samples was equal to the first integer and for recovery samples it was the original integer plus 0.1.

Scheme 1.3 Regions where bin area is averaged

2.925 → 2.915
2.445 → 2.425
2.415 → 2.395
2.235 → 2.195
2.105 → 2.075
1.995 → 1.905
1.385 → 1.335
1.125 → 1.055

All spectra submitted by an individual subject had the average of reference phase spectra for that person deducted from each of their samples, i.e. the spectra were reference phase standardized on a person by person basis, thus presenting only the change which had occurred for each person since the start of intervention. It has been found that this reduces noise in the data and improves the models formed.

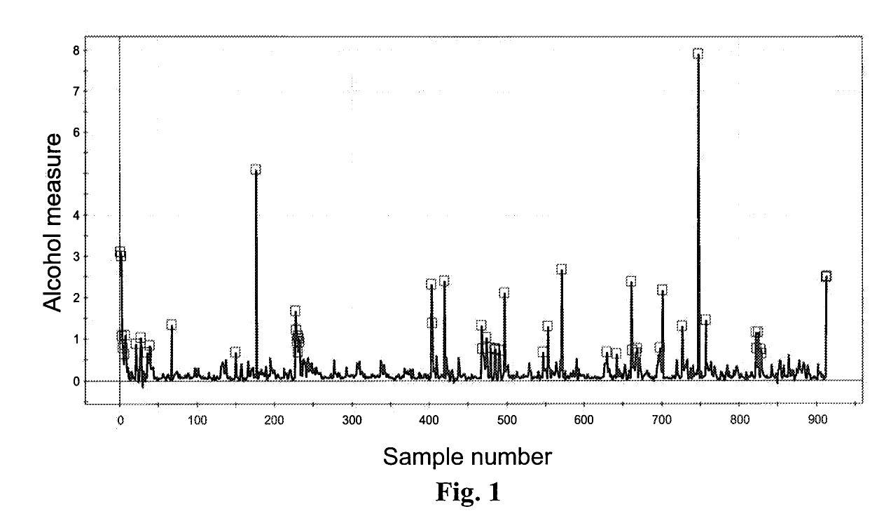

Principal components analysis (PCA) was then run on all of the NMR data to find outliers (centred scaling applied to all bins). Samples which were significantly over 3 standard deviations in the DModX or were abnormally high on the Hotelling's T² were removed as were those with levels of ethanol (shown by the methyl group at 1.2 ppm) significantly above reference phase levels (see FIG. 1). Outliers may be caused by the presence of food or toothpaste components indicating that the panellist has not collected true wake up saliva. It is also possible that the sample was allowed to degrade between collection and submission. The presence of food, drink, toothpaste or alcohol is easy to identify; degraded samples typically possess anomalously high lactate levels. The level of ethanol varies from person to person and from day to day. Ethanol is produced by some bacteria found in the mouth and may also carry over from beverages consumed the previous day/night. The highest levels are likely to be from those who have used a mouthwash before giving a sample; this may be a breach of the protocol justifying their immediate removal. The discarded samples were recorded together with the justification.

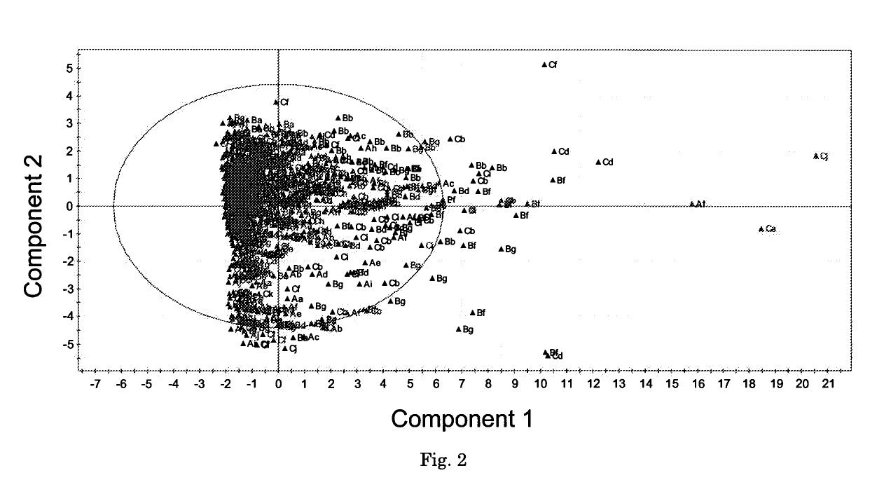

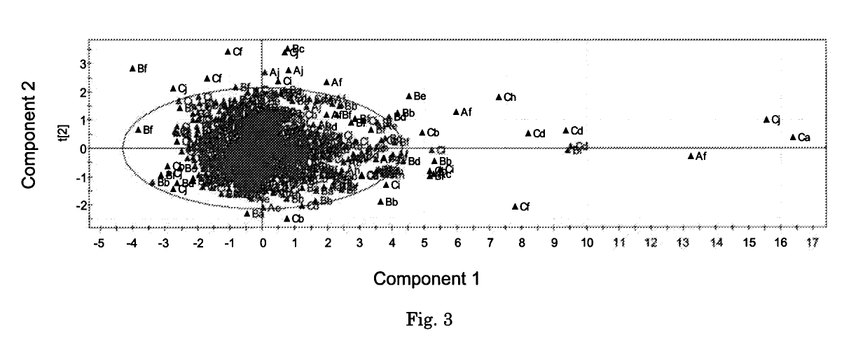

The result of the PCA analysis can be shown as a distribution along the first two principle components (first shown on the horizontal axis and second on the vertical) as shown in FIG. 2 or FIG. 3 which characterise the same set of data but without and with reference phase standardization being applied. Each data point is labelled with an identifier comprising an upper case letter (A, B, or C) indicating the product leg and a lower case letter indicating the individual on that leg. All the data have been normalised between 3.1 and 0.7 ppm to 100 area units. Acetate has been removed because it dominates the spectrum in this region and has been found not to provide useful distinguishing information. FIG. 3 illustrates the effect of reference phase standardisation. Lactate is the second largest peak in the differential spectra in this region and its variation strongly influences the spread of samples to the right along the first principal component axis in FIG. 2. In FIG. 3 this skew is all but lost when reference phase values are subtracted and the difference between the reference phase and the intervention phase is analysed. The first two components typically account for about 60% of all variance in the data.

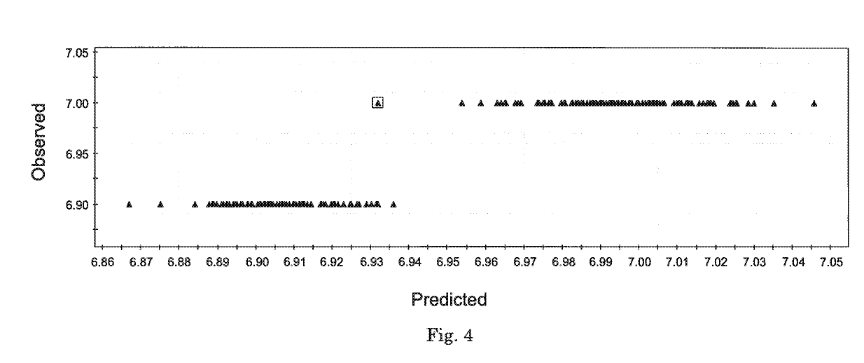

Once the data had been pruned for outliers each of the product legs (A, B, and C) was analysed separately to identify a ‘Mode of Action’ vector which distinguished the reference phase spectra from the intervention phase spectra. This was done by removing all of the recovery phase data and setting each product leg as a different class. An Orthogonal Partial Least Squares (O-PLS) analysis was then run for all classes using the difference of 0.1 in the phase identifier as the Y variable. FIG. 4 shows the plot of the observed vs. predicted spread. In this plot the algorithm seeks to gain maximum separation between samples identified as being from the reference phase from those identified as being from the intervention phase. Reference phase samples should be to the left of 6.95 whereas intervention phase samples should appear to the right. Only one value (highlighted) fails in this regard. Models were tested for predictivity by removing a third of the subjects from the model, building it, then predicting for the third removed based on the model the other two thirds produced. This was carried out for each of three random thirds chosen and the statistics of prediction determined based on them all. In this study the model built gave a 76% correct classification.

The Mode of Action vector for each product was taken as the loadings of the O-PLS first component. This was used qualitatively to determine what metabolites were increased or reduced by the intervention of each product. In the case of Product C, it was found that lactate levels tended to increase whilst propionate and butyrate levels tended to decrease. Product B was found to increase lactate and succinate but reductions in propionic or butyric were not significant. Product A showed little of significance; though lactate appeared to increase, the error was large and the change was not statistically significant.

### EXAMPLE 2

### Velocity of Action Investigation

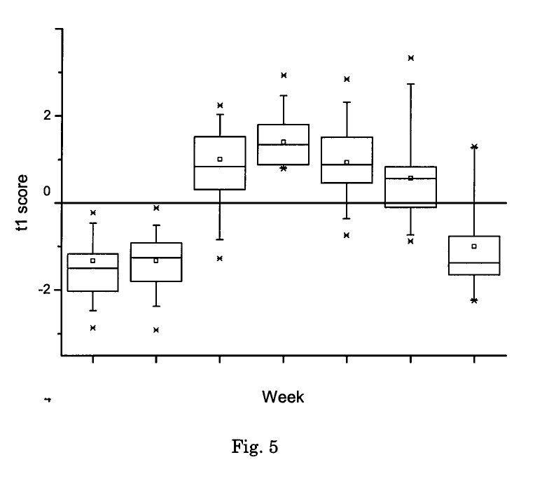

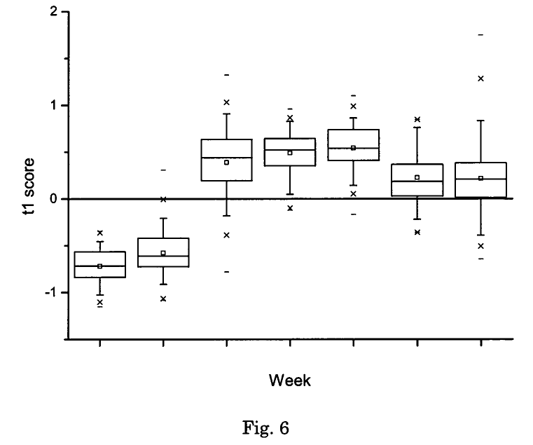

Building upon the work from Example 1, to determine the Velocity of Action (VoA) of a product the scores plot from the O-PLS was used. Data were batched by week for each phase (reference, intervention and recovery) and a box plot drawn for each batch in order on the same axes. Recovery phase data were obtained by projecting recovery samples into the model built in order to see the return to reference phase levels from the end of intervention. The plots for Product C and a further test product are shown in FIGS. 5 and 6. In these plots the weeks of the three product usage phases are shown along the x axis. Labels B1 and B2 show the two reference phase weeks, W1-W3 the intervention weeks and R1 and R2 the ‘recovery’ weeks. Plots for each product could only be viewed independently since they were all built on different models i.e. their y axes are different. They were however compared qualitatively for the nature of the retention of effect, the speed to plateau and the size of error bars. If the products have similar modes of action one could in principle use a common PLS component axis and compare them directly with one another. The product plotted in FIG. 6 shows a better retention of effect than Product C (FIG. 5) which, however, reaches its peak effect in the second week whereas the product of FIG. 6 takes three weeks to reach its maximum effect.

Such plots could be used to support e.g., comparative advertising but can also be used to design better studies where panellists are re-used (e.g. in a crossover study) so that a sufficiently long wash-out period is allowed between treatments.

### EXAMPLE 3

### Health Correlation

In order to link salivary metabonomics to clinical effects a number of the panellists on a trial were graded for signs of gingivitis, periodontitis and other symptoms (see Scheme 3.1 below). The result was a series of indices and one overall health score calculated in accordance with Scheme 3.1.

Scheme 3.1 Health scales

GI = Gingivitis Index (0-4)
PI = Plaque Index (0-4)
BPE = Basic Periodontal Exam. (0-6)
Calc = Calculus Index (0-3)
Tong = Tongue Coating (0-3)
Health = GI + PI + (2 × BPE) + Calc + Tong

The overall health score was correlated to bacterial metabolites as follows. Pre-processing and removal of outliers was carried out as in Example 1 but in this case only those samples which had been received in the same week as gradings were performed were taken. Each patient's samples for the grading week had the same clinical information attached and this was used as the set of y variables. Models were built to link metabolite levels to particular indices or to overall health. It was found that a correlation could be made to total health.

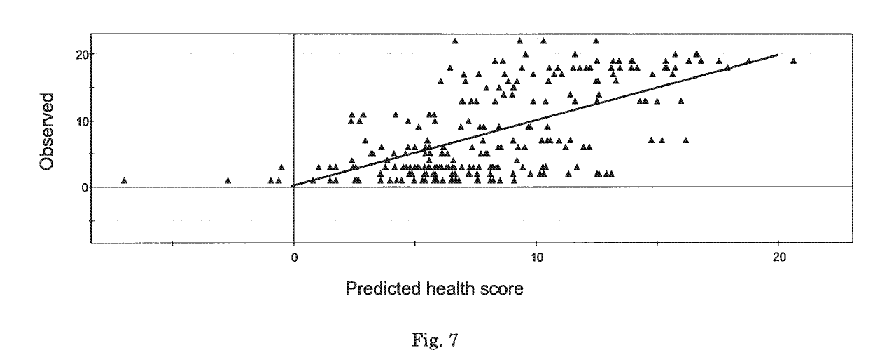

In order to correctly validate a model of this kind it is necessary to perform a similar prediction routine to that described in Example 1. Individuals are randomly assigned to one of three classes. In turn, the data from each one of the classes are set aside as a prediction set and a model is built from the remaining two classes. The prediction set is then placed into the model and, for these data points, an observed vs. predicted overall health scores plot drawn, as shown in FIG. 7. The three plots that result can all be combined and drawn on the same axes and the R² value, known as the root mean squared error of prediction (RMSEP), taken for a line of y=x (shown in FIG. 7).

### EXAMPLE 4

### Comparative Extent of Action

In order to convert Mode of Action (MoA), as discussed in Example 1, to Extent of Action (EoA) it was necessary to scale the MoA vectors to represent the magnitude of the change that had occurred. The loadings chart from the O-PLS model is produced as a particular type of unit vector known as an eigenvector. The corresponding eigenvalue of the eigenvector describes the magnitude of the vector or transformation. By multiplying each eigenvector by the corresponding eigenvalue from the model it is possible to scale them comparatively.

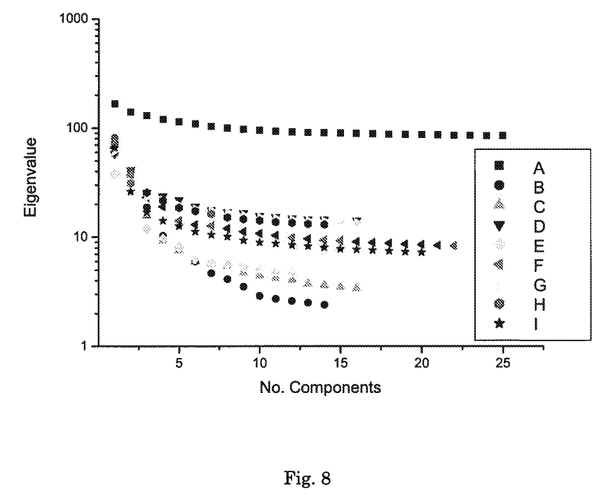

The eigenvalue from an O-PLS model is dependent on e.g., the separation displayed by the data, the dispersion of the points in each group being separated and the number of points in each group. It is also dependent on the number of components fitted to the data and this can vary greatly. As an O-PLS model is formed, successive additional components remove data not deemed to be explanatory and the amount of information on which the model is built decreases. Typically though, with each additional component the proportion of data that is removed decreases. The eigenvalue decreases with additional components but the differences between successive eigenvalues become progressively smaller. A dataset deriving from an underlying complex behaviour, but with little noise, may deliver a strong model including many components, each justified for inclusion but with decreasing additional explanatory value. Conversely, a dataset reflecting a lot of random noise may deliver a weak model having few components since the first few components remove a lot of data and successive components appear to make little improvement to the model. This has the effect that some of the weakest models appear to be the strongest, i.e. include fewer components, if the software is allowed to run unchecked. FIG. 8 shows the effect subsequent fitting of components has on the magnitude of eigenvectors from models built for a range of oral care treatment products, A-I. In this plot, products E and I are repeat runs based upon usage of the same commercial toothpaste, which corresponds to Product C in Example 1 and does not contain an antimicrobial agent. Likewise, products F and G are repeat runs based upon usage of the same triclosan-containing, commercial toothpaste, corresponding to Product A in Example 1. Product A is a commercially available mouth rinse containing chlorhexidine and, in this evaluation, was found to build the strongest model. Note that the y axis is logarithmic in order to better separate the different lines at low values.

For the methods herein the O-PLS models would generally be run until the difference between eigenvalueⁿ and eigenvalueⁿ⁺¹ was less than 0.1 (typical scale running from around 100 to 2) to ensure a stable eigenvalue. A result of this requirement is that a many components are fitted but the later ones are progressively less and less of the model. The important aspect though is not what has been removed but what has been kept. The information kept is only that which correlates to a difference between the reference and intervention phases. Three different approaches to the analysis were tried out:
1. All individuals on the same product leg were pooled together, with reference phase standardization. The model was built on the difference, for all samples involving that product use, between reference phase and intervention samples.
2. All individuals on the same product leg were pooled together, with reference phase standardization, but grouping the intervention phase samples by each of the three intervention weeks. Three models were built based on the difference between each of these weeks and the reference phase.
3. A model was built for each of the individuals in the trial based on the difference between all the intervention phase samples and the reference phase samples.

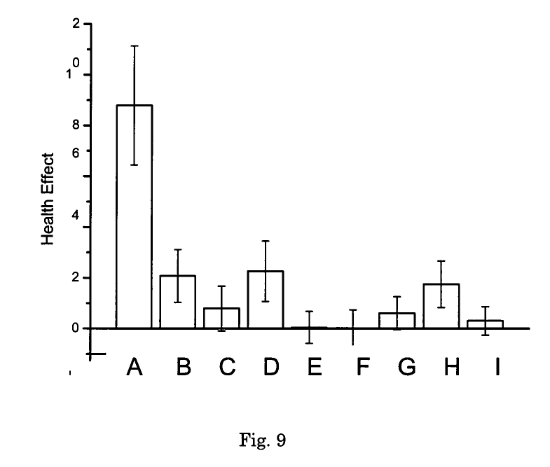

Once the models above had been built and scaled they were used as inputs into a PCA plot in five dimensions. The health correlation was scaled according to the average size of the other eigenvalues and was inserted in the positive (poor health) and negative (good health) form. The co-ordinates of the scores plot were taken and projected onto the health line so that each person or product had a score to show the amount of improvement, or deterioration, in overall oral health when moving from the reference phase to the intervention phase. The averages of these values by product, with 95% confidence intervals, are shown in FIG. 9 for approach 3 mentioned above.

It was also found that grouping people together at all (approaches 1 and 2) was undesirable as it assumed all people would behave in a similar way. Even when the reference phase standardisation is applied there is still a great difference in the effect experienced during intervention, perhaps from the different extents to which the panellists brush or conduct themselves in the intervention period. Best results were obtained when individual models were formed for each person and compared; this delivered the best statistical analysis and allowed t-tests of the groups to identify when a difference was statistically significant. In this example all the reference phase samples and all the intervention phase samples were included within the model with equal weights, with no differentiation applied as to when an intervention phase sample was taken. The net change is therefore a composite of the changes taking place throughout the whole of the three week intervention period. A more targeted estimate of the changes taking place after about three weeks product usage could be obtained by only including the third week's samples in the analysis. Of course an intervention period could be even longer, such as from 4 to 12 weeks, with sampling at the end of the intervention period.

FIG. 9 shows no significant difference between Products E and I or between F and G, which is to be expected since, as noted above, the products are the same in each case. Further since Product E/I was the product also being used in the reference (wash-out phase) a net improvement of zero, or non-significantly different from zero, is also to be expected.

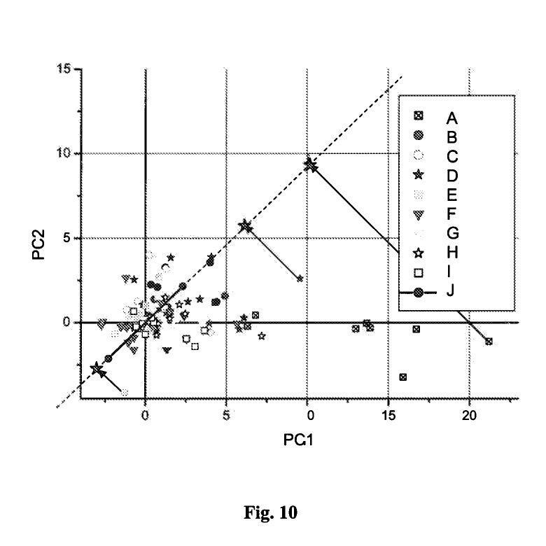

Each point on the plot of FIG. 10 represents the net change for an individual between reference phase and intervention phase in a two component space defined by the first two principle components (PC1 and PC2). The vector for improving overall oral health, as determined from the overall model, was also projected into this space and is represented by the dashed line shown. Though not accurately shown in FIG. 10, the health vector passes through the origin. As shown for three of the individuals, by projection onto the health vector the individuals' changes between the reference and intervention phases can be characterised as a movement along the health vector and a movement in a perpendicular direction not related to the underlying health measures.

Though the product usage in the foregoing examples involved systematic use of one product at a time only, the methodology also permits following a system of products involving flossing, brushes, mouthwashes and pastes and comparison between different systems of the same products using this method.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of computing a proxy oral health measure for an individual comprising the steps of:
a) collecting a saliva sample from the individual;
b) obtaining and digitising an individual spectrum from the individual's saliva sample;
c) comparing the digitised individual spectrum to a reference model stored in a computer memory to compute the proxy oral health measure, wherein the reference model is derived by correlating, through multivariate analysis, one or more direct measures of the oral health of each of a plurality of members of a reference population to reference spectra derived from saliva samples from the reference population members, the reference spectra corresponding in type to the individual spectrum.

2. The method according to claim 1 wherein the individual spectrum is a NMR spectrum, preferably a ¹H NMR spectrum.

3. A method according to claim 2 wherein the individual spectrum is a ¹H NMR spectrum and the comparison of the individual spectrum to the reference model comprises using that portion of the spectrum falling between 0.5-3.5 ppm, preferably 0.5-4.5 ppm, more preferably 0.5-8.6 ppm.

4. A method according to claim 3 wherein the portion used of each spectrum comprises the peaks for propionic acid, butyrate and trimethylamine.

5. A method according to claim 4 wherein the portion used of each spectrum further comprises the peaks for formate, N-acetyl sugars, lactate, methylamine, and dimethylamine.

6. A method according to claim 4 wherein the portion used of each spectrum further comprises one or more peaks selected from those methanol, trimethylamine oxide, phenyl-alanine, choline, histidine, tyrosine, methylguanidine, sarcosine, β-hydroxybutyrate, succinate, pyruvate, iso-butyrate, n-butyrate, leucine, alanine, n-valerate and ethanol.

7. A method according to claim 3 wherein the peak for acetate is removed from the analysis.

8. A method according to claim 1 wherein the saliva samples are obtained by having each individual rinse the oral cavity according to a standardised protocol and expectorate into a container, wherein, after expectoration of each saliva sample, the sample is treated with a stabiliser to prevent further bacterial metabolism of the sample.

9. A method according to claim 8 wherein each saliva sample is deep frozen after collection.

10. A method according to claim 1 wherein the one or more direct measures of the oral health of each of the members of the reference population are selected from:
a) a physician's quantitative assessment of oral health;
b) gingival images;
c) dental images; and
d) machine readings or expert assessment of breath malodour.

11. A method according to claim 10 wherein the physician's quantitative assessments of the population members comprise one or more indices selected from a plaque index, a calculus index, a gingival index, a periodontal index and a lingual furring index.

12. A method according to claim 1 wherein the reference model is constructed by PLS or O-PLS analysis of a data set comprising digital representations of the saliva spectra and the physician's quantitative assessments of the population members.

13. The use of a method according to claim 1 for estimating the individual's susceptibility to or degree of oral disease.

14. A method of generating an oral health history for an individual comprising providing a proxy oral health measure obtained according to the method of claim 1 from saliva samples collected on each of a plurality of days from the individual.

15. A method according to claim 14 wherein the history is generated in association with treating the subject with a test substance.

16. A method of selecting subjects for a clinical trial based upon the day-to-day consistency of their saliva composition as measured by the method of claim 1.

17. A method of selecting subjects for a clinical trial comprising the step of selecting the subjects from candidates for the trial based upon:
a) a proxy oral health measure for the candidates, obtained according to the method of claim 1; or
b) spectra obtained from saliva samples from each of the candidates.

18. A method according to claim 17 wherein the clinical trial comprises two or more legs and the subjects for each leg are chosen in order to balance the proxy oral health measure or metabolite levels of subjects across each of the legs, wherein the metabolite levels are determined from the individual spectra.

19. A method of managing a clinical trial comprising the steps of:
a) conducting a clinical trial on a set of individuals according to a predetermined protocol;
b) generating the oral health history for each of at least a sample of the individuals according to the method of claim 14;
c) examining the oral health histories thus obtained for indications of non-compliance with the clinical trial protocol.

20. A method of managing a clinical trial comprising the steps of:
a) recruiting a set of individuals who follow a predetermined protocol including a test or placebo oral treatment over a plurality of days;
b) requesting the individuals to sample their own saliva on one or more of the days and to return the saliva samples to a central collection point;
c) obtaining spectra from the samples after their return to the collection point; and
d) deriving one or more measures from the spectra selected from:
(i) data on the effectiveness of treatments applied to the individuals over the plurality of days; and
(ii) data on the day to day responses of individuals in the set.

21. A method of prescribing a treatment product for an individual comprising the step of examining the individual's proxy oral health measure provided by a method according to claim 1.

22. A method of determining the efficacy of a treatment product upon an individual comprising treating the individual with the treatment product and assessing the individual's oral health history, generated according to the method of claim 14, before and after treatment with the product.

23. A method of measuring the efficacy of a treatment product comprising the steps of:
a) conducting a clinical trial during which each of a set of subjects is treated with the treatment product and an oral health history is generated for each subject according to the method of claim 14; and
b) computing a product efficacy measure for the product from the oral health histories, or from product induced compositional changes in the saliva as determined from the spectra, for the set of subjects.

24. The method of claim 23 wherein the product efficacy measure is compared to that of a reference product.

25. The method according to claim 23 wherein the product treatment is effected after a period of normalising treatment.

26. The method according to claim 25 wherein samples of each subject's saliva are collected during the period of normalising treatment.

27. A method for generating advertising indicia for a treatment product comprising
a) measuring the efficacy of the treatment product according to the method of claim 23; and
b) associating the product efficacy measure with the product.

28. A method for generating advertising indicia for a treatment product comprising differentiating the mode of action of the product from that of a reference product by showing different product-induced compositional shifts in the trial subjects saliva.

29. A method of characterising a treatment product comprising the steps of:
a) collecting at least one starting saliva sample from each of a set of individuals;
b) treating the individuals with the treatment product;
c) collecting at least one end saliva sample from each of the individuals;
d) obtaining spectra from all of the saliva samples and storing the spectra in a database, each spectrum being associated with an individual identifier and with a sample type identifier;
e) performing a multivariate analysis upon the database of spectra to derive one or more treatment vectors associated with the effect of the treatment product upon the set of individuals.

30. A method according to claim 29 wherein at least one of the vectors describes a change in the set of individuals as a result of using the product.

31. A method according to claim 29 wherein at least one of the vectors differentiates a first subset of individuals from the whole set or from a second subset with respect to a response to the product.

32. A method according to claim 29 wherein the starting saliva samples are obtained before treatment of an individual with the treatment product.

33. A method according to claim 31 wherein the end saliva samples are obtained after treatment of an individual with the treatment product.

34. A method according to claim 32 wherein one or more intermediate saliva samples are obtained from the individual and further spectra derived from the intermediate saliva samples are stored in the database, associated with individual and sample type identifiers, and included in the multivariate analysis.

35. A method according to claim 34 wherein the intermediate saliva samples are obtained during treatment of an individual with the treatment product.

36. A method according to claim 29 wherein data from spectra from a plurality of an individual's starting saliva samples are averaged to provide a normalising measure for each individual and the normalising measure is subtracted from corresponding data for each of the individual's spectra before the multivariate analysis is performed.

37. A method of comparing two or more treatment products by comparing the treatment vectors associated with each product obtained according to the method of claim 29.

38. A method according to claim 37 wherein the multivariate analysis is a principle components analysis and the comparison comprises plotting each of the vectors in a space defined by one or more principle components.

39. A method according to claim 31 wherein a first subset of individuals is treated with a first treatment product, a second subset of individuals is treated with the first treatment product and a second treatment product, and the at least one vector differentiating the first subset from the second subset characterises a supplementary effect of the second treatment product with respect to the first treatment product.

40. A method according to claim 29 wherein the treatment product is an oral treatment product in the form of a toothpaste, a mouthwash, a denture adhesive, or a mechanical oral treatment device.

41. A method according to claim 40 wherein the oral treatment product includes an antimicrobial agent.
